(19) United States
(12) Patent Application Publication
(10) Pub. No.: US 2010/0191115 A1
(43) Pub. Date: Jul. 29, 2010

(54) ULTRASOUND IMAGING SYSTEM AND METHOD

(75) Inventor: Stefan Denk, Ried (AT)

Correspondence Address: PETER VOGEL;GE HEALTHCARE, 20225 WATER TOWER BLVD., MAIL STOP W492, BROOKFIELD, WI, 53045, US

(73) Assignee: General Electric Company, Schenectady, NY (US)

(21) Appl. No.: 12/358,481

(22) Filed: Jan. 23, 2009

(51) Int. Cl. A61B 8/14

(52) U.S. Cl. 600/447

(57) ABSTRACT

In one embodiment, a method of transmitting ultrasonic energy is provided. The method comprises steps of configuring a pulse generator for generating and supplying excitation signals, transmitting ultrasound energy, based on the excitation signals, into an image volume of interest and generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector and wherein at least a first transmit beam set and a last transmit beam set, in each sector, are not generated from neighboring beam positions.

### FIELD OF INVENTION

This invention generally relates to ultrasound imaging systems, and, more particularly, to a system and method for performing volumetric ultrasound imaging using multiple transmit beams.

### BACKGROUND OF THE INVENTION

Various noninvasive diagnostic imaging modalities are capable of producing cross-sectional images of organs or vessels inside the body. An imaging modality that is well suited for such real-time noninvasive imaging is ultrasound. Ultrasound diagnostic imaging systems are in widespread use by cardiologists, obstetricians, radiologists and others for examinations of the heart, a developing fetus, internal abdominal organs and other anatomical structures. These systems operate by transmitting waves of ultrasound energy into the body, receiving ultrasound echoes reflected from tissue interfaces upon which the waves impinge, and translating the received echoes into structural representations of portions of the body through which the ultrasound waves are directed.

It is possible to generate three-dimensional ultrasound images by either physically sweeping a one-dimensional array or using a two-dimensional array transducer to steer the transmitted and received ultrasound about two axes. Three-dimensional real-time imaging poses two major challenges: first, acquiring echoes from a volume at a sufficient sample density and in a sufficiently short time to maintain a real-time image frame rate, and, second, rendering high-resolution volumetric data obtained from these echoes to a suitable viewing format with sufficient speed to provide real-time display.

One method suggested in the prior art to improve the image frame rate is employing multiple transmit beams. Generating multiple transmit beam comprises “simultaneously” (within a few microseconds) emitting plurality of focused ultrasound pulses from an ultrasound transducer.

An area of interest is imaged using plurality of image frames. Each image frame is built up from plurality of transmit beam sets wherein each transmit beam set comprises multiple transmit beams. The sequence in which transmit beams are generated constitute a scan sequence. A general scan sequence comprises generating transmit beams from sequential (consecutive) transmit positions by electronically steering the ultrasound transducer. The general scan sequence suffers from the drawback that at certain scan positions two consecutive transmit beams from the generated image are separated by a large time gap.

Considering, there are twelve beam positions numbered from one to twelve, two simultaneous transmit beams may be generated at beam positions one and seven in parallel, subsequently at two and eight, and so on, until a final set of transmit beams are shot from beam positions six and twelve in parallel. The problem associated with this approach is that there exists a large time gap between the generation of transmit beams at positions six and seven. The time gap can be estimated to be six times t, where t is the time taken to complete a single transmit and receive shot. Understandably, the time gap becomes much larger for images with more scan positions.

The time gap observed in transmitting and receiving the transmit beams generated from two adjacent beam positions may contribute to causing undesired motion artifacts. Moreover, this time gap turns into a spatial gap for three-dimensional (3D) and/or four-dimensional (4D) scans employing a motor to physically move the ultrasound transducer to scan in different dimensions.

Hence there exists a need to provide an ultrasound imaging method that eliminates the disadvantages encountered in the prior art.

### BRIEF DESCRIPTION OF THE INVENTION

The above-mentioned shortcomings, disadvantages and problems are addressed herein which will be understood by reading and understanding the following specification.

In one embodiment, an ultrasound imaging system comprising a pulse generator for generating and supplying excitation signals, a transmitter coupled to the pulse generator for transmitting ultrasound energy into an image volume of interest, a transmit beamformer coupled to the transmitter, the transmit beamformer configured for generating multiple transmit beam sets from at least one sector comprising plurality of beam positions grouped into plurality of sub sectors, a receiver for receiving ultrasound echos reflected from the image volume of interest in response to the ultrasound energy transmitted, the receiver further configured for generating receive signals representative of the received ultrasound echos, a receive beamformer coupled to the receiver, the receive beamformer configured for processing receive signals to form at least one receive beam for each of the transmit beam and a processor coupled to the receive beamformer and operative to form an ultrasound image of the image volume in response to the receive beams is provided. Each sub sector comprises at least one set of beam positions indexed sequentially and each transmit beam set comprises multiple simultaneous transmit beams. The multiple simultaneous transmit beams are generated from beam positions with matching indexes in each sub sector. Further, at least two consecutive transmit beam sets are generated from beam positions not indexed sequentially.

In another embodiment, a method of acquiring ultrasonic data is provided. The method comprises steps of configuring a pulse generator for generating and supplying excitation signals, transmitting ultrasound energy, based on the excitation signals, into an image volume of interest, generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector and wherein at least two consecutive transmit beam sets are generated from beam positions not indexed sequentially, receiving ultrasound echos reflected from the image volume of interest in response to the ultrasound energy transmitted and further generating receive signals representative of the received ultrasound echos, processing receive signals to form at least one receive beam for each of the transmit beams and forming an ultrasound image of the image volume in response to the receive beams.

In another embodiment, a method of transmitting ultrasonic energy is provided. The method comprises steps of configuring a pulse generator for generating and supplying excitation signals, transmitting ultrasound energy based on the excitation signals, into an image volume of interest, generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector and wherein at least a first transmit beam set and a last transmit beam set, in each sector, are not generated from neighboring beam positions.

In yet another embodiment, a processor configured for electronically steering ultrasound imaging is provided. The processor comprises a routine for configuring a pulse generator for generating and supplying excitation signals, a routine for transmitting ultrasound energy, based on the excitation signals, into an image volume of interest, a routine for generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector and wherein at least two consecutive transmit beam sets are generated from beam positions not indexed sequentially, a routine for receiving ultrasound echos reflected from the image volume of interest in response to the ultrasound energy transmitted, a routine for generating receive signals representative of the received ultrasound echos, a routine for processing receive signals to form at least one receive beam for each of the transmit beam and a routine for forming an ultrasound image of the image volume in response to the receive beams.

Systems and methods of varying scope are described herein. In addition to the aspects and advantages described in this summary, further aspects and advantages will become apparent by reference to the drawings and with reference to the detailed description that follows.

### BRIEF DESCRIPTION OF THE DRAWINGS

FIG. 1 shows a block diagram of an ultrasound imaging system as described in one embodiment;

FIG. 2 illustrates scan sequence of an imaging frame with twelve beam positions, using two parallel transmit beams as described in one embodiment of the invention;

FIG. 3 shows a condensed notation of the scan sequence shown at FIG. 2;

FIG. 4 shows a schematic diagram of a sector comprising n sub sectors, as described in one embodiment of the invention; and

FIG. 5 illustrates a flow chart depicting a method of acquiring ultrasound data, as described in one embodiment of the invention.

### DETAILED DESCRIPTION OF THE INVENTION

In the following detailed description, reference is made to the accompanying drawings that form a part hereof, and in which is shown by way of illustration specific embodiments, which may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

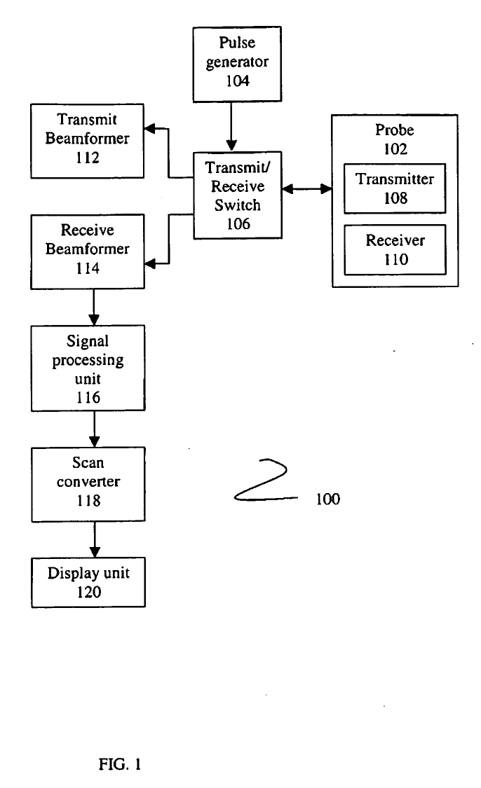

FIG. 1 shows an ultrasound imaging system 100 that directs ultrasound energy pulses into an object, typically a human body, and creates an image of the body based upon the ultrasound energy reflected from the tissue and structures of the body.

The ultrasound imaging system 100 comprises a probe 102 that includes a transducer array having plurality of transducer elements. The transducer array can be one-dimensional (1-D) or two-dimensional (2-D). A 1-D transducer array comprises plurality of transducer elements arranged in a single dimension and a 2-D transducer array comprises plurality of transducer elements arranged across two dimensions namely azimuthal and elevation. The number of transducer elements and the dimensions of transducer elements may be the same in the azimuthal and elevation directions or different. Further, each transducer element can be configured to function as a transmitter 108 or a receiver 110. Alternatively, each transducer element can be configured to act both as a transmitter 108 and a receiver 110.

The ultrasound imaging system 100 further comprises a pulse generator 104 and a transmit/receive switch 106. The pulse generator 104 is configured for generating and supplying excitation signals to the transmitter 108 and the receiver 110. The transmitter 108 is configured for transmitting ultrasound beams, along plurality of transmit scan lines, in response to the excitation signals. The term “transmit scan lines” refers to spatial directions on which transmit beams are positioned at some time during an imaging operation. The receiver 110 is configured for receiving echoes of the transmitted ultrasound beams. The transmit/receive switch 106 is configured for switching transmitting and receiving operations of the probe 102.

The ultrasound imaging system 100 further comprises a transmit beamformer 112 and a receive beamformer 114. The transmit beamformer 112 is coupled through the transmit/receive (T/R) switch 106 to the probe 102. The transmit beamformer 112 receives pulse sequences from the pulse generator 104. The probe 102, energized by the transmit beamformer 112, transmits ultrasound energy into a region of interest (ROI) in a patient's body. As is known in the art, by appropriately delaying the waveforms applied to the transmitter 108 by the transmit beamformer 112, a focused ultrasound beam may be transmitted.

The probe 102 is also coupled, through the T/R switch 106, to the receive beamformer 114. The receiver 110 receives ultrasound energy from a given point within the patient's body at different times. The receiver 110 converts the received ultrasound energy to transducer signals which may be amplified, individually delayed and then accumulated by the receive beamformer 114 to provide a receive signals that represents the received ultrasound levels along a desired receive line (“transmit scan line” or “beam”). The receive beamformer 114 may be a digital beamformer including an analog-to-digital converter for converting the transducer signals to digital values. As known in the art, the delays applied to the transducer signals may be varied during reception of ultrasound energy to effect dynamic focusing. The process of transmission and reception is repeated for multiple transmit scan lines to create an image frame for generating an image of the region of interest in the patient's body.

In an alternative system configuration, different transducer elements are employed for transmitting and receiving. In that configuration, the T/R switch 106 is not included, and the transmit beamformer 112 and the receive beamformer 114 are connected directly to the respective transmit or receive transducer elements.

The receive signals from the receive beamformer 114 are applied to a signal processing unit 116, which processes the receive signals for enhancing the image quality and may include routines such as detection, filtering, persistence and harmonic processing. The output of the signal processing unit 116 is supplied to a scan converter 118. The scan converter 118 creates a data slice from a single scan plane. The data slice is stored in a slice memory and then is passed to a display unit 120, which processes the scan converted data so as to display an image of the region of interest in the patient's body.

In one embodiment, high resolution is obtained at each image point by coherently combining the receive signals thereby synthesizing a large aperture focused at the point. Accordingly, the ultrasound imaging system 100 acquires and stores coherent samples of receive signals associated with each receive beam and performs interpolations (weighted summations, or otherwise), and/or extrapolations and/or other computations with respect to stored coherent samples associated with distinct receive beams to synthesize new coherent samples on synthetic scan lines that are spatially distinct from the receive scan lines and/or spatially distinct from the transmit scan lines and/or both. The synthesis or combination function may be a simple summation or a weighted summation operation, but other functions may as well be used. The synthesis function includes linear or nonlinear functions and functions with real or complex, spatially invariant or variant component beam weighting coefficients. The ultrasound imaging system 100 then in one embodiment detects both acquired and synthetic coherent samples, performs a scan conversion, and displays or records the resulting ultrasound image.

Ultrasound data is typically acquired in frames, each frame representing a sweep of an ultrasound beam emanating from the face of the transducer array. A 1-D transducer array produces 2-D rectangular or pie-shaped sweeps, each sweep being represented by a series of data points. Each of the data points are, in effect, a value representing the intensity of an ultrasound reflection at a certain depth along a given transmit scan line. On the other hand, the 2-D transducer array allows beam steering in two dimensions as well as focus in the depth direction. This eliminates the need to physically move the probe 102 to translate focus for the capture of a volume of ultrasound data to be used to render 3-D images.

One method to generate real-time 3-D scan data sets is to perform multiple sweeps wherein each sweep is oriented in a different scan plane. The transmit scan lines of every sweep are typically arrayed across the probe's 102 “lateral” dimension. The planes of the successive sweeps in a frame are rotated with respect to each other, e.g. displaced in the “elevation” direction, which is typically orthogonal to the lateral dimension. Alternatively, successive sweeps may be rotated about a centerline of the lateral dimension. In general, each scan frame comprises plurality of transmit scan lines allowing the interrogation of a 3-D scan data set representing a scan volume of some pre-determined shape, such as a cube, a sector, frustum, or cylinder.

In one exemplary embodiment, each scan frame represents a scan volume in the shape of a sector. Therefore the scan volume comprises multiple sectors. Each sector comprises plurality of beam positions, which may be divided into sub sectors. Each sub sector may comprise equal number of beam positions. However, it is not necessary for the sub sectors to comprise equal number of beam positions. Further, each sub sector comprises at least one set of beam positions and each beam position in a set of beam positions is numbered in sequence. Therefore, each sector comprises multiple sets of beam positions indexed sequentially on a predetermined rotation.

Plurality of transmit beam sets are generated from each sector. Further, each transmit beam set comprises one or more simultaneous transmit beams depending on the capabilities of the ultrasound imaging system 100. The term “simultaneous transmit beams” refers to transmit beams that are part of the same transmit event and that are in flight in overlapping time periods. Simultaneous transmit beams do not have to begin precisely at the same instant or to terminate precisely at the same instant. Similarly, simultaneous receive beams are receive beams that are acquired from the same transmit event, whether or not they start or stop at precisely the same instant.

The transmit beams in each transmit beam set are separated by plurality of transmit scan lines wherein each transmit scan line is associated with a single beam position. Thus, the multiple transmit beams are arranged in space separated such that they do not have significant interference effects.

The transmit beamformer 112 can be configured for generating each transmit beam set from beam positions having the same index value. Thus, beam positions with matching index value, in each sub sector, can be used for generating multiple simultaneous transmit beams that form a single transmit beam set. In one embodiment, at least two consecutive transmit beam sets are generated from beam positions not indexed sequentially. In an alternative embodiment, at least a first transmit beam set and a last transmit beam set, in a sector, are not generated from neighboring beam positions.

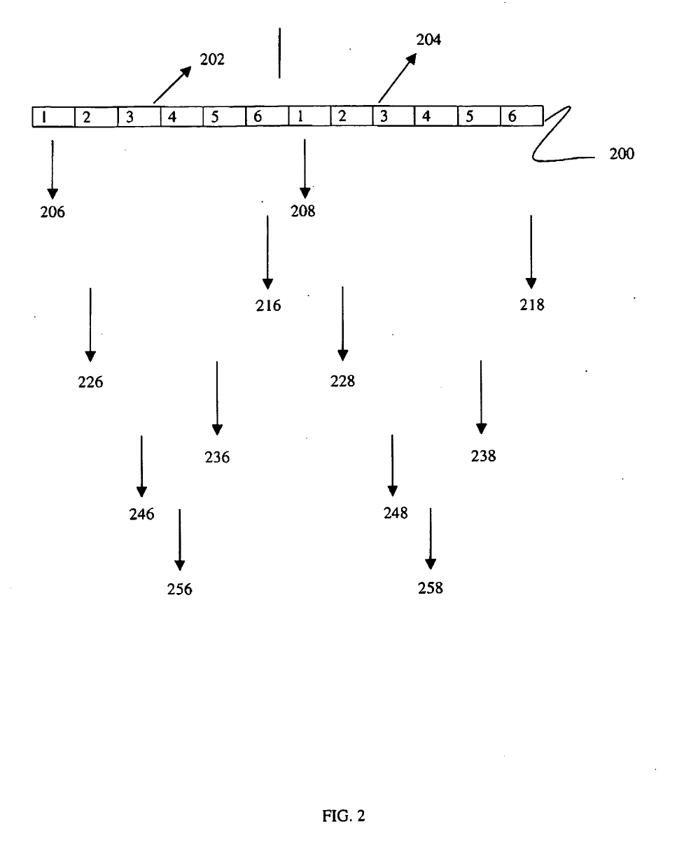

In an exemplary embodiment shown in FIG. 2, a scan sequence is provided for generating the transmit beams and/or transmitting ultrasound energy. A sector 200 comprising sub sectors 202 and 204 is shown schematically. Each of the sub sectors 202 and 204 are shown as comprising six beam positions indexed sequentially from one to six. A first transmit beam set comprising two simultaneous transmit beams 206 and 208 is generated from beam positions indexed as one. Similarly, a second transmit beam set comprising simultaneous transmit beams 216 and 218 is generated from beam positions indexed as six, a third transmit beam set comprising simultaneous transmit beams 226 and 228 is generated from beam positions indexed as two, a fourth transmit beam set comprising simultaneous transmit beams 236 and 238 is generated from beam positions indexed as five, a fifth transmit beam set comprising simultaneous transmit beams 246 and 248 is generated from beam positions indexed as three and a sixth transmit beam set comprising simultaneous transmit beams 256 and 258 is generated from beam positions indexed as four.

As can be understood from FIG. 2, the receive beams corresponding to each transmit beam have been omitted for brevity. Typically, 206, 216, 226, 236, 246 and 256 are directed along a different direction than 208, 218, 228, 238, 248 and 258 and the reference numerals are intended only to signify the relative order of the transmit beams within a transmit beam set and not the absolute spatial direction of the transmit beams. Taken as a whole, the transmit beam sets of each sector are spatially distributed to scan the region of interest in both elevation and azimuth.

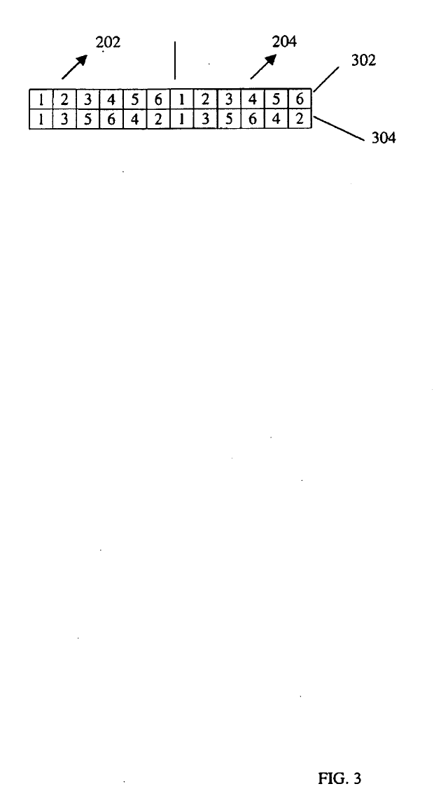

The condensed notation of this scan sequence is shown in FIG. 3. The row 302 shows numbering of beam positions in each sub sector 202 and 204, and the row 304 depicts the numbering of the transmit beam set of which, the transmit beam generated from the beam position indexed by the corresponding number in the first row, forms a part of. Skilled artisans shall however appreciate that dividing the beam positions in any sub sector into two substantially equal parts further provides a valid scan sequence.

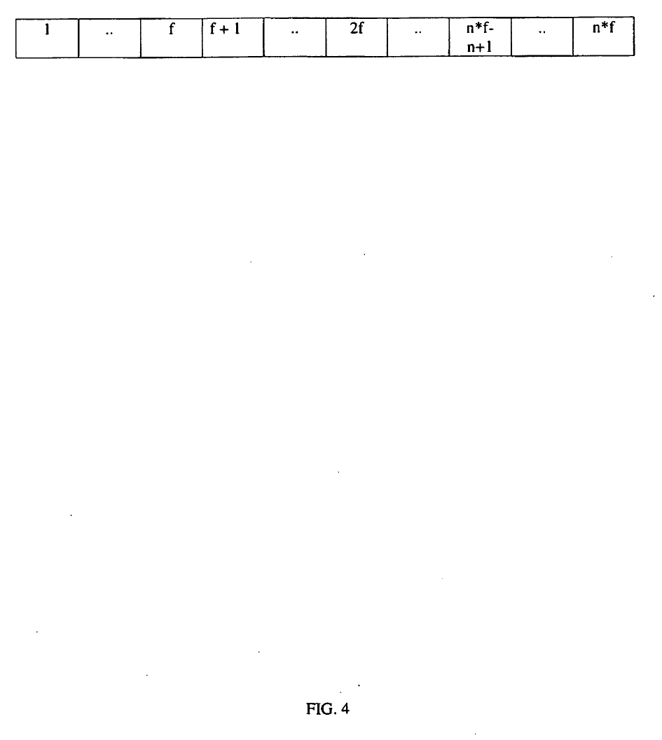

FIG. 2, describes an exemplary embodiment of the ultrasound imaging system 100 configured to generate six transmit beam sets from a sector comprising twelve beam positions which are grouped into two sub sectors, each comprising six beam positions indexed within the range of one to six. Further each of the six transmit beam sets is shown as comprising two simultaneous transmit beams generated in parallel. Skilled artisans shall however appreciate that in practice a sector can comprise any number of sub sectors. The beam positions in each sub sector may vary and further each transmit beam set may comprise any number of simultaneous transmit beams including one. This is further explained in conjunction with FIG. 4.

Considering each sector comprises ‘n’ sub sectors, as shown in FIG. 4, each transmit beam set comprises ‘n’ simultaneous transmit beams in parallel, considering ‘n’ as a positive integer. Further, each sub sector may comprise ‘f’ transmit scan lines. As each transmit scan line is associated with a single beam position, a set of ‘f’ beam positions may be grouped into a sub sector. Therefore, each sector to be scanned can be covered by n*f transmit scan lines. Within each sub sector the beam positions are provided relative to a predetermined position in the sub sector, for example, sub sector start position.

A formula representing a time of transmitting ultrasound beams at each beam position (one to ‘f’) within a sub sector (identical for all sub sectors) can be provided. This can be referred to as “shot time sequence”.

for f even (f=2*k):1, 3, . . . , 2*k−1, 2*k, 2*k−2, . . . , 2

and for f odd (f=2*k+1):1, 3, . . . , 2*k+1, 2*k, 2*k−2, . . . , 2

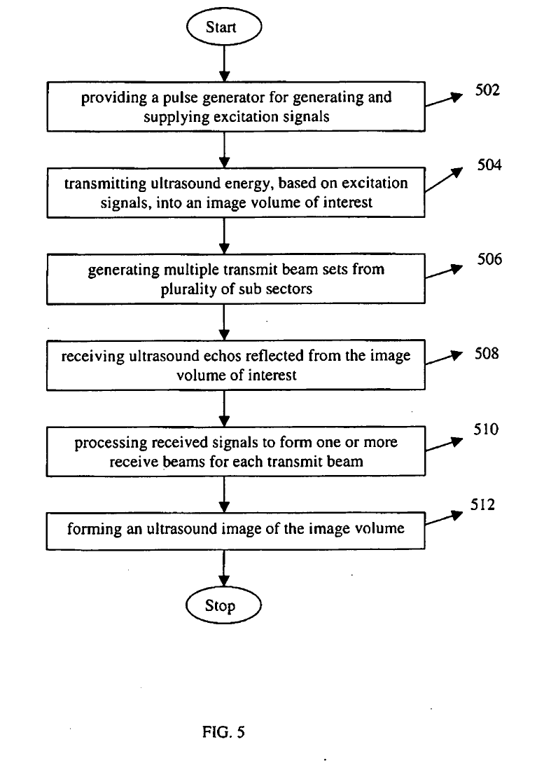

FIG. 5 flowcharts a method performed by the ultrasound imaging system 100 of FIG. 1. The method comprises steps of providing the pulse generator 104 for generating and supplying excitation signals to the transducer array at step 502, transmitting ultrasound energy, based on the excitation signals, into a region of interest at step 504 and generating multiple transmit beam sets from at least one sector comprising plurality of beam positions grouped into plurality of sub sectors at step 506. Each transmit beam set comprises multiple simultaneous transmit beams generated from beam positions with matching indexes in each sub sector and at least two consecutive transmit beam sets are generated from beam positions not indexed sequentially.

Returning to FIG. 5, ultrasound echos reflected from the image volume of interest are received by the receive beamformer 114 at 508. These ultrasound echos are processed to acquire multiple receive beam sets, at 510. In one embodiment, a single receive beam is acquired in response to each of the transmit beams, along the same direction as the respective transmit beam. In another embodiment, multiple receive beams are acquired from each transmit beam. The round trip delay time is shortest for those targets closest to the ultrasound transducer array, and longest for those targets farthest from the transducer array.

Further, a real-time, medical diagnostic ultrasound image is formed at 512 by the signal processing unit 116 in response to the receive beams described above. The ultrasound image is then displayed on the display unit 120. The term “real-time” means that the ultrasound image is displayed to a user during an ultrasound imaging session in which the images are obtained shortly following the acquisition of image data. The displayed ultrasound image is one of a two-dimensional (2-D) and a three-dimensional image (3-D). The term “3-D image” is intended broadly to encompass any image formed from a 3-D data set, including sectional views and various types of renderings and projections, for example.

The ultrasound imaging system 100 set forth in the invention is specifically constructed for the purpose, i.e. ultrasound imaging. However, the methods recited herein may operate on a general purpose computer or other network device selectively activated or reconfigured by a routine stored in the computer and interfaced with the ultrasound imaging system 100. The procedures presented herein are not inherently related to any particular ultrasound imaging system, computer or other apparatus. In particular, various machines may be used with methods in accordance with the teachings herein, or it may prove more convenient to construct more specialized apparatus to perform the desired method steps.

Accordingly, in one embodiment a processor configured for electronically steering the ultrasound imaging is provided. The processor comprises a routine for configuring a pulse generator 104 for generating and supplying excitation signals, a routine for transmitting ultrasound energy, based on the excitation signals, into an image volume of interest, a routine for generating multiple transmit beam sets from at least one sector comprising plurality of beam positions grouped into plurality of sub sectors, each transmit beam set comprising multiple simultaneous transmit beams, the multiple simultaneous transmit beams being generated from beam positions with matching indexes in each sub sector, wherein at least two consecutive transmit beam sets are generated from beam positions not indexed sequentially.

The processor further comprises a routine for receiving ultrasound echos reflected from the image volume of interest in response to the ultrasound energy transmitted, a routine for generating receive signals representative of the received ultrasound echos, a routine for processing receive signals to form at least one receive beam for each of the transmit beams and a routine for forming an ultrasound image of the image volume in response to the receive beams.

Some of the advantages of the ultrasound system and method of ultrasound imaging described in various embodiments of the invention are described below.

The scan sequences described herein minimizes the undesired motion artifacts resulting from the time gap observed in receiving the transmit beams generated from two consecutive beam positions at the interface of two sub sectors and subsequently, resulting spatial gaps in 3-D/4-D image acquisition.

Multiple transmit beams are generally employed to increase the frame rate. A transmit beam set comprising “n” simultaneous transmit beams increases the frame rate by “n” times.

The embodiments described above can be used in any suitable ultrasound imaging mode, including for example tissue harmonic imaging, contrast harmonic imaging, B-mode imaging, color Doppler imaging, spectral doppler imaging, and frequency dependent focus imaging.

In various embodiments of the invention, a method for ultrasound imaging and an ultrasound imaging system using the method are described. However, the embodiments are not limited and may be implemented in connection with different applications such as blood flow imaging and heart imaging. The application of the invention can be extended to other areas, for example non-destructive evaluation of materials, such as castings, forgings, or pipelines using ultrasound. The invention provides a broad concept of using a scan sequence to generate multiple transmit beams, which can be adapted in a similar imaging system. The design can be carried further and implemented in various forms and specifications.

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An ultrasound imaging system comprising:
a pulse generator for generating and supplying excitation signals,
a transmitter coupled to the pulse generator for transmitting ultrasound energy into an image volume of interest;
a transmit beamformer coupled to the transmitter, the transmit beamformer configured for generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector;
a receiver for receiving ultrasound echos reflected from the image volume of interest in response to the ultrasound energy transmitted, the receiver further configured for generating receive signals representative of the received ultrasound echos;
a receive beamformer coupled to the receiver, the receive beamformer configured for processing receive signals to form at least one receive beam for each of the transmit beams; and
a processor coupled to the receive beamformer and operative to form an ultrasound image of the image volume in response to the receive beams;
wherein at least two consecutive transmit beam sets are generated from beam positions not indexed sequentially.

2. The system of claim 1, wherein the ultrasound image is a two dimensional image.

3. The system of claim 1, wherein the ultrasound image is a three dimensional image.

4. The system of claim 1, further comprises a scan converter and a display unit.

5. The system of claim 1, wherein the transmit beams in each transmit beam set are separated by plurality of transmit scan lines, each transmit scan line being associated with a single beam position.

6. A method of acquiring ultrasonic data, the method comprising:
configuring a pulse generator for generating and supplying excitation signals;
transmitting ultrasound energy, based on the excitation signals, into an image volume of interest;
generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector;
receiving ultrasound echos reflected from the image volume of interest in response to the ultrasound energy transmitted;
generating receive signals representative of the received ultrasound echos;
processing receive signals to form at least one receive beam for each of the transmit beams; and
forming an ultrasound image of the image volume in response to the receive beams;
wherein at least two consecutive transmit beam sets are generated from beam positions not indexed sequentially.

7. The method of claim 6, wherein each sub sector comprises n beam positions.

8. The method of claim 7, wherein a first transmit beam set is generated from beam positions indexed first in each sub sector and a second transmit beam set is generated from beam positions indexed n in each sub sector.

9. The method of claim 7, wherein a third transmit beam set is generated from beam positions indexed second in each sub sector and a fourth transmit beam set is generated from beam positions indexed n−1 in each sub sector.

10. The method of claim 7, wherein a fifth transmit beam set is generated from beam positions indexed third in each sub sector and a sixth transmit beam set is generated from beam positions indexed n−2 in each sub sector.

11. The method of claim 7, wherein the transmit beams in each transmit beam set are separated by plurality of transmit scan lines, each transmit scan line being associated with a single beam position.

12. The method of claim 6, wherein the ultrasound image is a two dimensional image.

13. The method of claim 6, wherein the ultrasound image is a three dimensional image.

14. A method of transmitting ultrasonic energy, the method comprising:
configuring a pulse generator for generating and supplying excitation signals;
transmitting ultrasound energy based on the excitation signals, into an image volume of interest; and
generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector;
wherein at least a first transmit beam set and a last transmit beam set, in each sector, are not generated from neighboring beam positions.

15. The method of claim 14, wherein the transmit beams in each transmit beam set are separated by plurality of transmit scan lines, each transmit scan line being associated with a single beam position.

16. The method of claim 14, wherein the multiple simultaneous transmit beams of at least one of the transmit beam sets are directed in separate respective directions distributed in three spatial dimensions.

17. A processor configured for electronically steering ultrasound imaging, the processor comprising:
a routine for configuring a pulse generator for generating and supplying excitation signals;
a routine for transmitting ultrasound energy, based on the excitation signals, into an image volume of interest;
a routine for generating multiple transmit beam sets from at least one sector comprising plurality of beam positions grouped into plurality of sub sectors, each sub sector comprising at least one set of beam positions indexed sequentially based on a predetermined rotation and wherein each transmit beam set comprises multiple simultaneous transmit beams, the multiple simultaneous transmit beams being generated from beam positions with matching indexes in each sub sector;
a routine for receiving ultrasound echos reflected from the image volume of interest in response to the ultrasound energy transmitted;
a routine for generating receive signals representative of the received ultrasound echos;
a routine for processing receive signals to form at least one receive beam for each of the transmit beams; and
a routine for forming an ultrasound image of the image volume in response to the receive beams;
wherein at least two consecutive transmit beam sets are generated from beam positions not indexed sequentially.

18. The processor of claim 17, wherein each sub sector comprises n beam positions.

19. The processor of claim 18, wherein a first transmit beam set is generated from beam positions indexed first in each sub sector and a second transmit beam set is generated from beam positions indexed n in each sub sector.

20. The processor of claim 18, wherein a third transmit beam set is generated from beam positions indexed second in each sub sector and a fourth transmit beam set is generated from beam positions indexed n−1 in each sub sector.

21. The processor of claim 18, wherein a fifth transmit beam set is generated from beam positions indexed third in each sub sector and a sixth transmit beam set is generated from beam positions indexed n−2 in each sub sector.

22. The processor of claim 18, wherein the transmit beams in each transmit beam set are separated by plurality of transmit scan lines, each transmit scan line being associated with a single beam position.

23. The processor of claim 17, wherein the ultrasound image is a two dimensional image.

24. The processor of claim 17, wherein the ultrasound image is a three dimensional image.
